(19) United States
(12) Patent Application Publication
(10) Pub. No.: US 2006/0240054 A1
(43) Pub. Date: Oct. 26, 2006

(54) Irritant-free gel compositions

(76) Inventors: Bernard D. Graney, Hoosick Falls, NY (US); Otto A. Weil, Bennington, VT (US)

Correspondence Address: HESLIN ROTHENBERG FARLEY & MESITI PC, 5 COLUMBIA CIRCLE, ALBANY, NY, 12203, US

(21) Appl. No.: 11/359,607

(22) Filed: Feb. 22, 2006

Related U.S. Application Data
(60) Provisional application No. 60/655,073, filed on Feb. 22, 2005.

(51) Int. Cl. A61K 8/73; F41C 9/00

(52) U.S. Cl. 424/401; 42/1.08

(57) ABSTRACT

Irritant-free gel compositions according to the present invention include less than 5 parts by weight of one or more marking agents, 0.25-2 parts by weight of a thickening agent; and 30-90 parts by weight water. The thickening agent is typically a vegetable gum. In some embodiments, the compositions may additionally include 20-60 parts by weight of at least one water-miscible solvent, such as monohydric alcohols, polyhydric alcohols and mixtures thereof. The water-miscible solvent may be methyl alcohol, ethyl alcohol, isopropyl alcohol, n-propyl alcohol, ethylene glycol, propylene glycol or a mixture thereof, and preferably a mixture of isopropyl alcohol and propylene glycol.

### CROSS-REFERENCE TO RELATED APPLICATIONS

This application is a non-provisional of, and claims priority from, U.S. Provisional Application No. 60/655,073, filed Feb. 22, 2005, the entire disclosure of which is incorporated herein by reference.

### BACKGROUND OF THE INVENTION

Personal defense sprays incorporating chloroacetophenone (CN) or o-chloro-benzalmalononitrile (CS) teargas are used by police and military personnel to subdue unruly or disruptive persons, and by private citizens to ward off attackers and/or to repel animals such as dogs and bears without inflicting long-term or permanent damage. Typically sprays incorporating the aforementioned chemicals are very irritating and cause short-term pain to be experienced by a person exposed to the sprays. Sprays based on non-irritating or inert gel compositions have the advantage that they do not cause irritation or pain. Yet they still can be sprayed toward a subject's eyes; allowing shortly thereafter, the subject to be subdued without harm and/or allow a potential victim to escape without permanent physical injury. Inert gel compositions are particularly attractive for use by law enforcement or private citizens in countries that outlaw the possession of defense sprays that contain irritants.

### SUMMARY OF THE INVENTION

An aspect of the present invention relates to an irritant-free gel composition comprising: less than 5 parts by weight of one or more marking agents; 0.25-2 parts by weight of a thickening agent; and 30-90 parts by weight water.

A second aspect of the present invention relates to an irritant-free gel composition consisting of essentially of: 0.01-5% of one or more marking agents; 0.25-2% xanthan gum; 20-60% mixture of isopropyl alcohol and propylene glycol; and 33-79.74% water.

A third aspect of the present invention relates to a method of crowd control and/or personal defense, the method comprising spraying toward a threatening person or animal an irritant-free gel composition described above.

A fourth aspect of the present invention relates to a method for manufacturing an irritant-free gel composition, the method comprising mixing less than 5 parts by weight of one or more marking agents, 0.25-2 parts by weight of a thickening agent and 20-60 parts by weight of a water-miscible solvent, and gradually adding water to form a gel.

A fifth aspect of the present invention relates to a device for marking a target individual comprising: a) a pressurized canister containing a propellant; and b) an irritant-free gel composition as described above.

### DETAILED DESCRIPTION OF THE INVENTION

The present invention relates to water based irritant-free/inert gel compositions containing less than 5 parts by weight of one or more marking agents, 0.25-2 parts by weight of a thickening agent and 30-90 parts by weights water. The irritant-free gel compositions may be sprayed at a target up to 25 feet away, a significant increase over conventional solvent-based sprays that travel only about 15 feet. The formulation is ideal for indoor use as cross-contamination is minimized, since the marking agent is contained in the gel which sticks to the affected area. The composition sticks to the target. Where the target is the face of an assailant, he may be temporarily blinded, allowing for safe detention by law enforcement or military officials and/or safe escape by potential victims. Because the composition is non-flammable, it may be used together with a stun gun, TASER® electronic weapons, and/or similar weapons without the danger of the electric or electronic weapons igniting a fire. Water miscibility allows for quick and easy decontamination of an assailant using just soap and water. The composition is irritant-free.

Irritants as the term is used herein, and as consistent with its usage in the art, (see Occupational Health and Safety Administration (OSHA) definition according to Appendix A 29 CFR 1910.1200) refers to materials or agents that are non-corrosive, but which cause a reversible inflammatory effect on living tissue by chemical action at the site of contact. The irritant-free gel compositions of the present invention do not contain, i.e. are free of, capsaicin, capsaicinoids and Oleoresin Capsicum (OC), or other irritants such as alphachloroacetophenone (CN), ortho-chlorobenzalmalononitrile (CS), bromobenzenecyanide (CA), dibenz-(b,f-1,4-oxazepine (CR), chloropicrin, benzene, and combinations thereof.

Marking agents are chemical compounds that leave a trace or sign that identify a person or subject that has a marking agent on them. A marking agent can be visible as in the case of dyes or colorants or invisible to naked eye in the case of ultra-violet (UV) dyes. UV dyes are visible upon exposure to long- or short-range UV radiation. The marking agents are used so that a person sprayed with the irritant-free gel composition may be identified visually at a later time under UV or visible light. An example of a visible marking agent includes but is not limited to FD&C red # 40. An example of an invisible marking agent includes but is not limited to Pylaklor UV dye. Typically the marking agents are non-toxic. The aforementioned marking agents are not meant to limit the types of marking agents that may be used in an embodiment of the present invention. Any water-soluble and/or water-dispersible UV dye, and staining or non-staining colorant or dye can be used in accordance with the present invention.

The amount of marking agents contained in the irritant-free gel compositions range from about 0.01 to about 5 parts by weight. The amount of marking agent used may vary in ranges with a lower limit 0.01, 0.5, 1, or 2 to an upper limit of 3, 4, or 5. All ranges are inclusive and combinable. Typically about 0.3 to about 2 parts by weight are used in the inert gel compositions.

Thickeners for the irritant-free gel composition are those that yield a sprayable, stable gel when used in the amount of 5% or less, typically 2% or less, based on the total weight of the composition. One or more cationic, nonionic, and/or anionic thickeners may be used; suitable materials are listed in the Glossary and Chapters 3, 4, 12 and 13 of the Handbook of Water-Soluble Gums and Resins, Robert L. Davidson, McGraw-Hill Book Co., New York, N.Y., 1980, incorporated by reference herein.

Natural thickeners are typically used, especially those that are suitable as food additives, such as vegetable gums and celluloses, such as carboxymethylcellulose, methylethylcellulose sodium carboxymethylcellulose, and hydroxyethylcarboxymethylcellulose. Other natural and synthetic gums and gum-like materials may be used if desired, including natural and synthetic inorganic clays, such as bentonite; silicas; and polymeric thickeners, such as acrylates, polyvinylacetate, and acrylamide. The thickeners are typically hydrated or gelled with water or alkanols, especially with polyhydric alcohols such as glycerol and sorbitol. Vegetable gum thickeners are particularly preferred. These include alginic acid, sodium alginate, potassium alginate, ammonium alginate, calcium alginate, propylene glycol alginate, agar agar, isinglass, carrageenan, processed eucheuma seaweed, arabinogalactan, locust bean gum, guar gum, tragacanth, acacia, karaya gum, and gellan gum. Xanthan gum is typically used.

The amount of thickener to be used depends on the amount and type of the other materials included in the formulation, especially the amount of water, and on the desired viscosity/rheology of the final product. Typically, it is desired that the product have a viscosity/rheology profile such that the product remains on vertical surfaces on which it is sprayed for at least a few seconds, and seeks its own level inside the container during spraying so that the container does not retain an appreciable amount of residue upon emptying. Typically, the amount of thickener used ranges from about 0.25-2 percent by weight. For compositions thickened with xanthan gum, amounts of thickener are typically 0.3% to 1%, preferably 0.5%-0.75%. For pH dependant thickeners, the amount and type of the thicker is based upon the pH of the composition. Modifiers, such as urea, sodium chloride, sodium sulfate, magnesium sulfate, ammonium chloride and magnesium chloride and combinations thereof, may be present in the compositions in addition to the thickener(s) if non-pseudoplastic compositions are desired or if pseudoplastic compositions are to be modified in their viscosity behavior

Irritant-free gel compositions according to the present invention may additionally include a water-miscible solvent in the amount of 20-60 parts by weight or 20%-60%, to prevent freezing. Food-grade solvents and/or those that are “generally regarded as safe” by the U.S. Federal Department of Agriculture are typically used. Water-miscible solvents are capable of being mixed with water in any concentration without separation of phases; e.g., water and ethyl alcohol are miscible. Water-miscible solvents that may be used in the compositions of the present invention include alcohols, such as methanol, ethanol, isopropanol, and n-propanol. Other suitable solvents include glycols, such as propylene glycol, ethylene glycol, hexylene glycol, dipropylene glycol, diethylene glycol, tripropylene glycol, triethylene glycol; glycol ethers such as glyme, diglyme; glycol ether PM, glycol ether EP, glycol ether PP, glycol ether PTB, glycol ether EB, glycol ether PB, glycol ether DPM, glycol ether DM, glycol ether DE, glycol ether DP, glycol ether DPP, glycol ether DB, glycol ether DPB, glycol ether TPM, and glycol ether TPB; esters, such as glycol ether DPM acetate, glycol ether DE acetate, and glycol ether DB acetate; ketones, such as acetone, methyl ethyl ketone, diacetone alcohol, and other solvents such as acetic acid, acetonitrile, dimethyl formamide, dimethyl sulfoxide, dioxane, glycerin, and tetrahydrofuran. Typical water-miscible solvents include monohydric alcohols, such as methyl alcohol, ethyl alcohol, isopropyl alcohol, n-propyl alcohol, or mixtures thereof; polyhydric alcohols, such as ethylene glycol, propylene glycol or mixtures thereof; and mixtures of monohydric and polyhydric alcohols. Typically the solvent is a mixture of isopropyl alcohol and propylene glycol.

In another aspect, the present invention relates to methods for crowd control and/or personal defense. The method may be used with people or animals that the operator or user of the irritant-free gel wishes to control or escape from. For best results, the composition is usually sprayed toward the subject's eyes; shortly thereafter, the subject may be subdued without harm and/or the potential victim may escape without permanent physical injury. Irritant-free gel compositions are particularly useful for crowd control and/or personal defense in countries that outlaw possession of personal defense sprays containing irritants. One example is the United Kingdom, where UK section 5(1)(b) of the Firearms 1968 Act prohibits possession of any tear gas, irritant projector or the like. An irritant-free gel composition, containing both a visible marking dye and an invisible UV marking dye, may be sprayed in an attacker's face to temporarily “blind” the attacker. The attacker is marked with a visible dye (red is typically used), and in addition, with an invisible UV dye which may retain detectable under a long- or short-range UV lamp for several days.

In another aspect, the present invention relates to a device for marking a target individual comprising: a) a pressurized canister containing a propellant; and b) an irritant-free gel composition as described above. The irritant-free gel composition of the present invention may be utilized in any number of conventional spray canisters or containers. U.S. Pat. No. 6,399,073 B2 to Pinkney discloses such a spray canister or container. The entire contents of which is incorporated herein by reference. Such canisters are well known in the art and they typically have a cavity for storing the irritant-free gel composition and a propellant such as nitrogen (N₂) gas for pressurizing the cavity.

In order to be properly utilized in a pressurized canister a carrier must be present as a liquid or gas at room or ambient temperature and one atmosphere of pressure. Typically the boiling point of the carrier is less than 225° F. Additionally, the carrier must not solidify or freeze at temperatures as low as −10° F. and typically as low as −20° F. Further, the carrier must be such that the components of the irritant-free gel composition do not separate or come out of solution over extended periods of time such as three days, three weeks, three months or even three years. The carrier described above is actually a component of the irritant-free gel composition (see below).

Typically the carrier is a mixture of propylene glycol, water, and anhydrous isopropanol. Including the carrier as a component of the irritant-free gel composition is not meant to limit the use of carriers in an embodiment of the present invention. A carrier separate from the irritant-free gel composition may used in accordance with the present invention if a potential carrier has the above performance characteristics. An example of device for marking a target individual is a pistol having adapted for use therein a pressurized canister containing a propellant and the irritant-free gel composition

Irritant-free gel compositions according to the present invention may be prepared by mixing the marking agents, typically in the form of a water-dispersible marking agent, thickening agent(s) and solvent(s), if solvents are to be used. Water is gradually added to form a gel. Any other emulsifiers are added before the water, usually with the marking agent. In an alternate process, the thickening agent is mixed with water initially and then the marking agents and solvent(s) are added.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

### EXAMPLES

### Procedure

1. Weigh and record the weight of an empty drum.
2. Add to the empty drum propylene glycol, anhydrous isopropanol (IPA), water-soluble FD&C red # 40, Tween 80, Pyaklor LX-10747 UV dye and Keizan RD.
3. Premix the above for 30 minutes.
4. Slowly add triple-filtered water while maintaining constant, moderate agitation using a high-shear, variable speed mixer. Once all of the water has been added the final product must be agitated for approximately one hour.
5. Mix the drum for one hour before use.

### Example 1

### Irritant-Free Gel Composition

| | | |
|---|---|---|
| 70 | lbs. | Triple-filtered water |
| 23 | lbs. | Propylene glycol |
| 5 | lbs. | Anhydrous (99%) isopropanol (IPA) |
| 0.3 | lbs. | FD&C red # 40 |
| 0.50 | lbs. | Pyaklor LX-I0747 Ultraviolet Dye |
| 1.2 | lbs. | Kelzan RD (xanthan gum) |
| 100.0 | lbs. | Total Net Weight |

What is claimed is:

1. An irritant-free gel composition comprising:
less than 5 parts by weight of one or more marking agents;
0.25-2 parts by weight thickening agent; and
30-90 parts by weight water.

2. An irritant-free gel composition according to claim 1, wherein the marking agent is a water-soluble or water-dispersible ultraviolet dye.

3. An irritant-free gel composition according to claim 1, wherein the marking agent is a water-soluble or water-dispersible colorant dye.

4. An irritant-free gel composition according to claim 1, wherein the thickening agent is vegetable gum.

5. An irritant-free gel composition according to claim 1, wherein the thickening agent is xanthan gum.

6. An irritant-free gel composition according claim 1, additionally comprising 20-60 parts by weight of at least one water-miscible solvent.

7. An irritant-free gel composition according to claim 1, wherein the at least one water-miscible solvent is selected from monohydric alcohols, polyhydric alcohols and mixtures thereof.

8. An irritant-free gel composition according to claim 7, wherein the at least one water-miscible solvent is selected from methyl alcohol, ethyl alcohol, isopropyl alcohol, n-propyl alcohol, ethylene glycol, propylene glycol and mixtures thereof.

9. An irritant-free gel composition according to claim 7, wherein the at least one water-miscible solvent is a mixture of isopropyl alcohol and propylene glycol.

10. An irritant-free gel composition consisting essentially of:
0.01-5% of one or more marking agents;
0.25-2% xanthan gum;
20-60% mixture of isopropyl alcohol and propylene glycol; and
33-79.74% water.

11. An irritant-free gel composition according to claim 10, wherein the marking agent is a water-soluble or water-dispersible ultraviolet dye.

12. An irritant-free gel composition according to claim 10, wherein the marking agent is a water-soluble or water-dispersible colorant dye.

13. A method of crowd control and/or personal defense, said method comprising spraying toward a threatening person or animal an irritant-free gel composition according to claim 1.

14. A method for manufacturing an irritant-free gel composition, the method comprising mixing less than 5 parts by weight of one or more marking agents, 0.25-2 parts by weight of a thickening agent and 20-60 parts by weight of a water-miscible solvent, and gradually adding water to form a gel.

15. A device for marking a target individual comprising:
a) a pressurized canister containing a propellant; and
b) an irritant-free gel composition of claim 1.

16. A device according to claim 15, wherein the device is a pistol.
